(19) United States
(12) Patent Application Publication
(10) Pub. No.: US 2011/0044909 A1
(43) Pub. Date: Feb. 24, 2011

(54) ANIMAL MODEL FOR DIABETES

(76) Inventor: Stephanie Tozzo, Pfeffingen (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 12/990,662

(22) PCT Filed: Apr. 27, 2009
(86) PCT No.: PCT/EP2009/055026

§ 371 Date: Nov. 2, 2010

(30) Foreign Application Priority Data
May 6, 2008 (EP) 08155736.5

(51) Int. Cl. A61K 49/00; A01K 67/00

(52) U.S. Cl. 424/9.2; 800/9

(57) ABSTRACT

The present invention provides a method of producing diabetic mice and their use in drug development.

The present invention relates to a method of producing a diabetic mouse which can be used in a method of identifying compounds that can reverse diabetes and are suitable for interventive therapy in diabetes and its complications.

The past decades have witnessed a dramatic increase in the prevalence of obesity and type-2-diabetes (T2D) primarily in US but also progressing in Europe and in developing countries. All over the world, T2D represents 85-90% of all the cases of diabetes (inherited insulin-dependent diabetes or T1D and non-insulin-dependent-diabetes Mellitus or T2D). Although several mutations affecting leptin pathway, insulin secretion or its receptor, or GLUT4 (glucose transport sensitive to insulin) have been identified, most of the cases of obesity and T2D are from non-genetic origin and triggered by life style (reduction of physical activity, highly calorie diets, etc). T2D and obesity are both associated with high morbidity, mortality and health care costs due to their micro- and macro-vascular complications.

The primary feature of T2D is insulin resistance and defect in insulin secretion. Insulin is a key hormone synthesized and secreted by pancreatic beta-cells that stimulates glucose uptake in various organs (particularly muscle, liver, and adipose tissue). Insulin also regulates Hepatic Glucose Production (HGP) via controlling the expression of the gene encoding glucose-6-phosphatase and inhibits lipolysis in adipose tissue. Impaired insulin action (i.e. insulin resistance) occurs when target tissues are unable to respond to normal concentrations of insulin.

Once this dysregulation starts and in absence of treatment, beta-cells secrete increased amount of insulin (=hyperinsulinemia) to maintain euglycemia (normal circulating glucose levels). However, in the absence of treatment, beta-cells fail producing enough insulin, leading to increase in circulating glucose (hyperglycaemia). As long as enough beta-cells will be viable and will be secreting the appropriate rate of insulin to maintain euglycemia, T2D does not arise. The mechanisms leading to insulin resistance have been extensively explored during the past years by many groups. It is widely accepted that the accumulation of free fatty acids (FFA) in insulin-sensitive non adipose tissues (liver & muscles), can impair insulin-mediated-glucose uptake in these tissues. Moreover, increased lipid production by the liver enhances fatty acid oxidation, decreases insulin-dependent inhibition of hepatic glucose production and, therefore, increases gluconeogenesis (GNG), further worsening the hyperglycaemia.

Development of T2D is associated with other metabolic disturbances. The cluster of insulin resistance, impaired glucose tolerance, arterial hypertension, abdominal obesity and dyslipidemia, called metabolic syndrome (Syndrome X), has been defined by the Adult Treatment Panel III (ATPIII) as a grouping of factors that underlie major cardiovascular risk. Many preclinical studies suggested a predominant role of insulin in the development of hypertension in T2D patients. However, the primary focus of clinical care is to diagnose and treat the abnormalities in glucose metabolism. Indeed high blood glucose is a major risk factor for microvascular complications as reported by the UK Prospective Diabetes Study (UKPDS, 1998). It was shown that maintenance of glucose levels near to normal in T2D patients prevents the onset of defects such as neuropathy (occurring in 50% to 60% of T2D patients), retinopathy and nephropathy (diabetes are the leading causes of blindness and end-stage renal failure in the U.S).

The anti-diabetic drugs from the thiazolidinediones (TZDs) class (available in the US since 1997), currently represented by Rosiglitazone (Avandia®), Pioglitazone (Actos®), and Troglitazone (Rezulin®), are efficacious drugs in increasing peripheral insulin-mediated-glucose-uptake. TZDs are pharmacological agonists of peroxisome-proliferator-activated receptor (PPAR-gamma), a transcription factor of the nuclear hormone receptor family that controls the expression of genes in glucose and lipid metabolism. PPAR-gamma drugs reduced hyperglycaemia, hyperlipidemia and hyperinsulinemia and improved insulin sensitivity by increasing differentiation and proliferation of pre-adipocytes into mature fat cells, particularly in peripheral fat depots. Thus PPAR-gamma activation increases fatty acids storage in peripheral adipocytes, lowers circulating fatty acids and reduces triglycerides levels in muscle and liver. PPAR-gamma drugs alter the expression of several circulating factors such as adiponectin, TNF alpha and resistin, the levels of which are highly correlated to insulin resistance and the response to therapy.

Appropriate animal models of T2D and insulin resistance are essential preclinical tools for characterizing in vivo efficacy of therapeutic agents. Most of the animal models of T2D that have been developed in the past 20 years are based on genetic mutations. Spontaneously diabetic (or insulin resistant and obese) rodent models such as, db/db and ob/ob mice, GK, ZDF and fa/fa rats are most commonly used worldwide in drug discovery.

Due to this rapid metabolic deterioration, reversion of main feature of T2D in ZDF rats is not achieved by marketed anti-diabetics (Rosiglitazone (PPAR-gamma) and Raziglitazar (PPAR alpha gamma)). Prevention of T2D was shown to be achieved, however, in ZDF rats under chronic treatment (13 weeks) (Shibata et al., 2000) or in very young and only moderately diabetic animals (Brand et al., 2003; Pickavance et al., 2005). It was reported by one team that in a slightly different T2D model called the VDF rat (Vancouver Diabetic Fatty), derived from Zucker and fa/fa strains, which was used by that interventional therapy with a DPPIV inhibitor was able to partially improve glucose tolerance, peripheral insulin sensitivity, and beta-cell function. It has to be noted that this model was not diabetic, being characterized by absence of hyperglycaemia, weak glucose intolerance and no peripheral insulin resistance.

It was the aim of the present invention to develop an animal model for insulin resistance, obesity, type 2 diabetes and its complications that overcomes at least some of the disadvantages of animal models described in the prior art, mainly the potential interference of a target pathway with the mutated pathway, i.e. leptin pathway or other.

It is an object of the present invention to provide a method of producing a non-human animal model for insulin resistance, obesity, type 2 diabetes. Said method comprises the steps of: feeding a mouse of the age of about 9 weeks for about 20 weeks with a high fat high sugar diet.

In a preferred embodiment the mouse is a male mouse.

In a further preferred embodiment the mouse is a C57BL/6J mouse.

In a further preferred embodiment the feeding of the high fat high sugar diet occurs for about 22 weeks.

In a further preferred embodiment the mouse is on a normal chow diet until the start of the feeding the high fat high sucrose diet.

In yet another preferred embodiment the chow diet comprises KLIBA 3436 diet. The detailed composition of KLIVA 3436 diet is given in FIG. 4.

In a further preferred embodiment the high fat high sugar diet is a custom made SSNIFF D12492 modified 60% Kcal fat diet. The detailed composition of SSNIFF D12492 modified 60% Kcal diet is given in FIG. 5.

In a second aspect the present invention provides a method for producing a non-human animal model for complications of type 2 diabetes, said method comprising feeding a mouse of the age of about 9 weeks for about 30 weeks with a high fat high sugar diet. Mice that have been fed for about 30 weeks with the high fat high sugar diet are a suitable animal model for insulin resistance, obesity, type 2 diabetes and type 2 diabetes complications.

In a preferred embodiment the feeding of the high fat high sugar diet occurs for about 32 weeks.

In a second aspect, the present invention provides a method for identifying and/or testing compounds for the treatment of type 2 diabetes and/or its complications. Said method comprises the steps of administering a compound of interest to a mouse produced by a method of the present invention and determining whether diabetes and/or at least one type 2 diabetes complication are reversed by said compound.

The term “type 2 diabetes complications” comprises diseases such as cardiovascular disease, retinopathy, neuropathy, nephropathy and NAFLD (non-alcoholic fatty liver disease).

In an exemplary embodiment of the method for identifying and/or testing compounds for the treatment of type 2 diabetes, a candidate compound is administered to a mouse which has been produced according to the present invention, and an indicator value (e.g. a blood glucose level, or an insulin level in blood or in other tissues) having a correlation with insulin resistance, obesity and/or type 2 diabetes is then measured in the mouse. Thereafter, the obtained indicator value is compared with that of a control animal. Based on the comparative results, it is confirmed whether or not the candidate compound is able to alleviate or eliminate the symptoms of type 2 diabetes. Specifically, the blood glucose level of a mouse of the present invention, to which a candidate compound has been administered, is measured. When the measured blood glucose level is lower than that of a control animal, which has not been in contact with the candidate compound, the candidate compound can be selected as a therapeutic agent for the treatment of type 2 diabetes.

Moreover, the insulin level of a mouse of the present invention, to which a candidate compound has been administered, can be measured as an indicator value having correlation with insulin resistance, obesity and/or type 2 diabetes. When the measured insulin level is lower than that of a control animal, which has not been in contact with the candidate compound, the candidate compound can be selected as a therapeutic agent for the treatment of type 2 diabetes.

The animal model of the present invention allows to test compounds for their ability to treat complications associated with type 2 diabetes such as e.g. nephrotoxicity and NAFLD, since the mice of the present invention develop complications associated with type 2 diabetes e.g. nephrotoxicity and NAFLD. For example, a test compound can be tested for its ability to treat type 2 diabetes as described above and for its ability to treat complications associated with type 2 diabetes by determining whether the compound can revert and/or alleviate type 2 diabetes complications developed by the animals of the present invention such as e.g. nephrotoxicity and NAFLD.

Examples of candidate compounds include a peptide, a protein, a non-peptide compound, a synthetic compound, a fermented product, a cell extract, a cell culture supernatant, a plant extract, a tissue extract and blood plasma of mammal (e.g. a mouse, a rat, a swine, a bovine, a sheep, a monkey, a human, etc.). Such compounds may be either novel compounds or known compounds. These candidate compounds may form salts. Examples of such salts of candidate compounds include salts with physiologically acceptable acids (e.g. organic acids and inorganic acids, etc.) or with bases (e.g. metal salts, etc.). In particular, physiologically acceptable acid-addition salts are preferable. Examples of such salts include salts with inorganic acids (e.g. hydrochloric acid, phosphoric acid, hydrobromic acid, sulfuric acid, etc.), or salts with organic acids (e.g. acetic acid, formic acid, propionic acid, fumaric acid, maleic acid, succinic acid, tartaric acid, citric acid, malic acid, oxalic acid, benzoic acid, methanesulfonic acid, benzenesulfonic acid, etc.).

In a third aspect, the present invention provides a use of a mouse of the present invention for the identification and/or testing of compounds for the treatment of type 2 diabetes and/or its complications.

### SHORT DESCRIPTION OF THE FIGURES

FIG. 1 shows the body weight of mice fed with high fat/sucrose (HF-HS diet) vs. mice fed with chow diet. It can be seen that high fat/sucrose fed mice develop obesity,

FIG. 2 shows the blood glucose concentration of high fat/sucrose (HF-HS diet) fed mice vs. chow diet fed mice. It can be seen that high fat/sucrose (HF-HS diet) fed mice develop fasting hyperglycemia,

FIG. 3 shows fasting plasma insulin levels of mice after 19-22 weeks on special diet vs. chow diet (DIO=diet induced obesity). It can be seen that high fat/sucrose (HF-HS diet) fed mice develop fasting hyperinsulinemia,

FIG. 4 shows the composition of KLIBA 3436 diet,

FIG. 5 shows the composition of Ssniff™ EF M D12492 mod. 60% fat energy diet,

FIG. 6a shows H & E staining of liver of 42 week old DIO mice (high fat diet). It can be seen that high fat/sucrose (HF-HS diet) fed mice develop severe hepatic steatosis,

FIG. 6b shows H & E staining of liver of a 25 week old chow diet fed mice (control),

FIG. 7a shows Sirius red staining of liver of 42 week old DIO mice (high fat diet),

FIG. 7b shows Sirius Red staining of liver of 25 week old chow diet fed mice (control), It can be seen that high fat/sucrose (HF-HS diet) fed mice develop severe hepatic fibrosis,

FIG. 7c shows Trichrome Masson staining of liver of 42 week old DIO mice (high fat diet). It can be seen that high fat/sucrose (HF-HS diet) fed mice develop severe hepatic steatosis,

FIG. 8a shows H & E staining of kidney of 42 week old DIO mice (high fat diet). It can be seen that high fat/sucrose (HF-HS diet) fed mice develop severe glomerular hypertophy and mesangial expansion,

FIG. 8b shows H & E staining of kidney of a 25 week old chow diet fed mice (control),

FIG. 8c shows H & E staining of kidney of 42 week old DIO mice (high fat diet). It can be seen that high fat/sucrose (HF-HS diet) fed mice develop severe glomerular sclerosis,

FIG. 8d shows H & E staining of kidney of a 25 week old chow diet fed mice (control),

FIG. 8e shows Trichrome Masson staining of kidney of 42 week old DIO mice (high fat diet). It can be seen that high fat/sucrose (HF-HS diet) fed mice develop severe renal inflammation and

FIG. 8f shows Trichrome Masson staining of kidney of 25 week old chow diet fed mice (control).

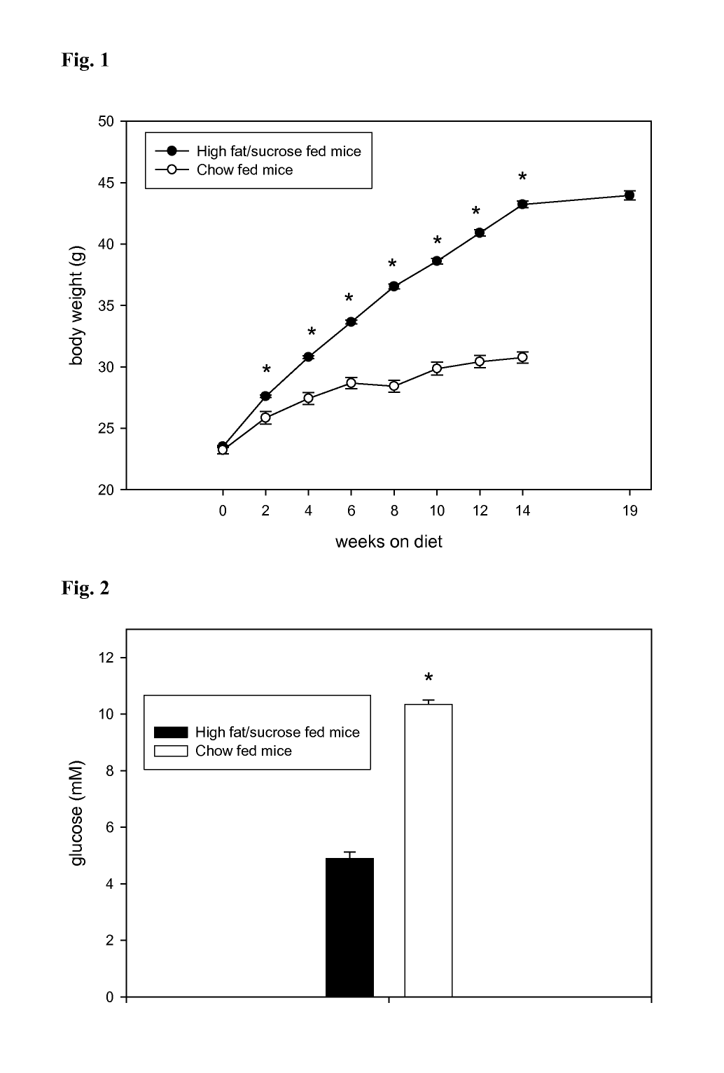

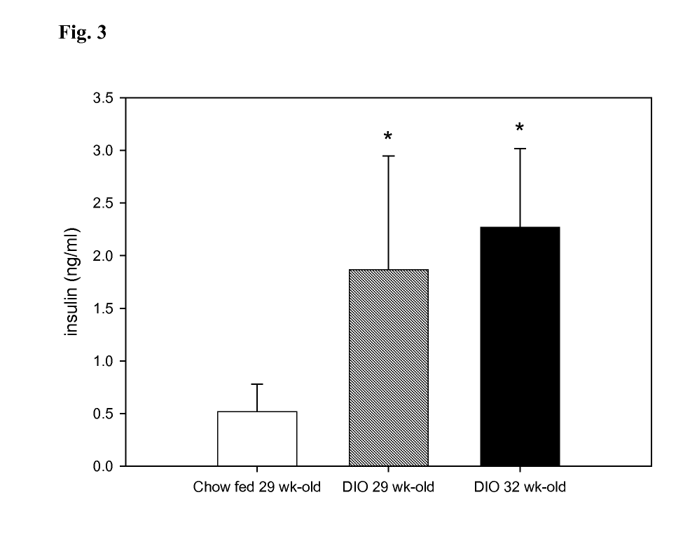

### EXPERIMENTAL PART

### Diabetes and Obesity Induction in Mice

Providers of mice: Charles River Germany
Background: Male C57BL/6J mice
Housing conditions: Standard-12 hrs light cycle,
Diet: Normal chow diet until the age of 9 weeks, then animals put on High Fat High Sucrose (HF-HS) (Energy: 60% from fat, 20% from sugar—SSNIFF D12492 mod.) for 14 weeks and transferred to Roche at the age of 23 weeks.
Adaptation period: in Roche for 6-20 weeks, animals maintained on HF-HS diet
Lean Control animals: Group of 10 animals kept under standard chow diet KLIBA 3436
Animals ready for experiments at the age of 29-31 weeks or 42 weeks for the study of diabetic complications
HF-HS diet maintained during the experimental phase

NASH in DIO Mice

Nonalcoholic fatty liver disease (NAFLD) is estimated to affect at least one quarter of the United States population and is the most common cause of abnormal liver function. NAFLD probably results from dysregulation of hepatic lipid metabolism as a component of the metabolic syndrome, which is collectively manifested as visceral obesity, dyslipidemia, atherosclerosis, and insulin resistance. Despite a benign prognosis for many cases, NAFLD can progress to nonalcoholic steatohepatitis (NASH), liver fibrosis, cirrhosis, and hepatocellular carcinoma, and a comprehensive understanding of the mechanism(s) of NAFLD-to-NASH transition remains elusive. Abnormality in lipid metabolism accompanied by chronic inflammation is a central pathway for the development of atherosclerosis and NASH. Indeed, a “two-hit” theory has been proposed, and the identification of secondary susceptibility factors related to lipid and cholesterol metabolism and inflammation that precipitate NASH in NAFLD patients is an important step in the treatment of this disease. Susceptibility to NAFLD and NASH progression may include genetic variation, infection, environmental exposure, and dietary contribution, which may lead to chronic inflammation and/or oxidative stress.

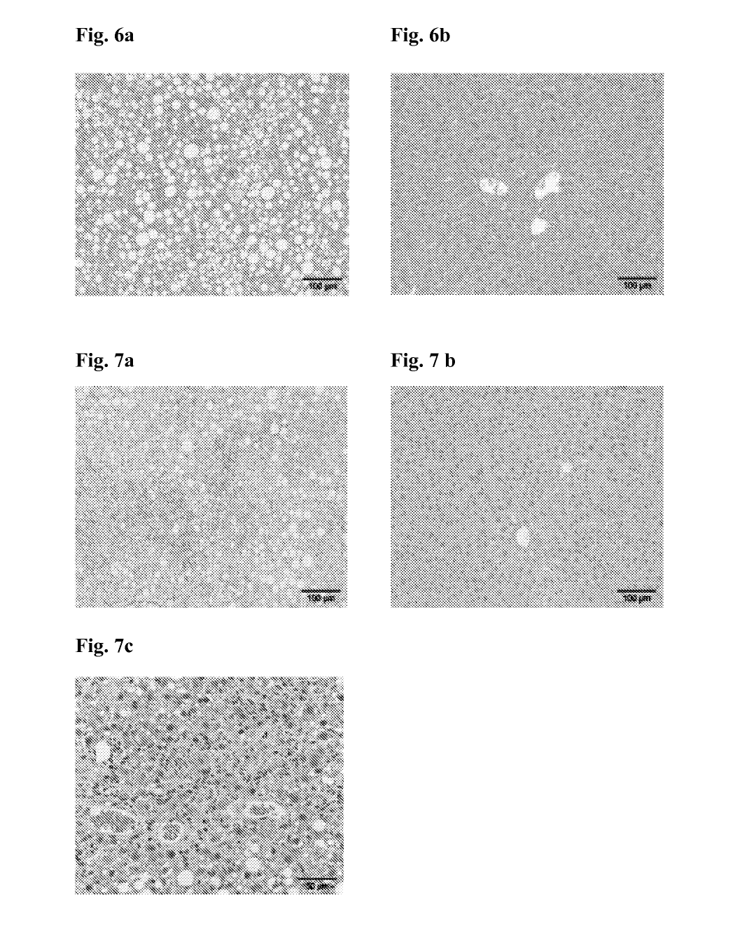

The following lesions were observed by histological evaluation in livers of 42 week old DIO mice vs. 25 week old DIO mice and 25 week old chow fed lean mice (see FIGS. 6 and 7):
Moderate to severe diffuse macrovacuolar hepatocellular vacuolation which correlated with diffuse macrovacuolar lipid deposits (confirmed by fat stain) (FIG. 6a vs. FIG. 6b)
Minimal to slight bile duct and Kupffer cell hyperplasia (FIG. 6a vs. FIG. 6b)
Minimal to marked infiltration of mononuclear inflammatory cells (FIG. 6a vs. FIG. 6b)
Minimal to moderate fibrosis (confirmed by Sirius Red and Trichrome Masson stain) (FIGS. 7a and 7c vs. 7b)

TABLE 1
NASH in 42 wk-old DIO mice
Incidence of relevant liver changes in DIO mice

| | 25 wk old | 25 wk old | 42 wk old |
|---|---|---|---|
| | chow | DIO | DIO |
| Bile duct hyperplasia | 0/10 | 0/10 | 7/10 |
| Kupffer cell hyperplasia | 0/10 | 0/10 | 5/10 |
| Lipid deposits, macrovacuolar, | 0/10 | 8/10 | 7/10 |
| diffuse | | | |
| Infiltration, mononuclear | 5/10 | 7/10 | 8/10 |
| Fibrosis | 1/10 | 2/10 | 10/10 |

Diabetic Nephropathy in DIO Mice

Diabetic nephropathy, is a progressive kidney disease caused by angiopathy of capillaries in the kidney glomeruli. It is characterized by nephrotic syndrome and nodular glomerulosclerosis. It is due to longstanding diabetes mellitus, and is a prime cause for dialysis in many Western countries. As type 2 diabetes is nowadays occurring at much earlier ages it is also expected that patients will develop nephropathy earlier that the usual older age, between 50 and 70 years old. The disease is progressive and may cause death two or three years after the initial lesions, and is more frequent in men. Diabetic nephropathy is the most common cause of chronic kidney failure and end-stage kidney disease in the United States. People with both type 1 and type 2 diabetes are at risk. The risk is higher if blood-glucose levels are poorly controlled. Further, once nephropathy develops, the greatest rate of progression is seen in patients with poor control of their blood pressure. Also people with high cholesterol level in their blood have much more risk than others. Diabetic nephropathy continues to get gradually worse. Complications of chronic kidney failure are more likely to occur earlier, and progress more rapidly, when it is caused by diabetes than other causes. Dialysis may be necessary once end-stage renal disease develops. At this stage, a kidney transplantation must be considered. Even after initiation of dialysis or after transplantation, people with diabetes tend to do worse than those without diabetes.

The goals of treatment are to slow the progression of kidney damage and control related complications. The main available treatment, once proteinuria is established, is ACE inhibitors and angiotensin receptor blockers, which usually reduce proteinuria levels and slow the progression of the disease but have several issues. The key issue is that despite an effective treatment, the residual risk for ESRD (End Stage Renal Disease) in patients with diabetic nephropathy remains high (˜16%). If a new drug can provide a substantial relative risk reduction by delaying glomerulosclerosis this will also result in a major absolute risk reduction.

There is a great medical need for medications that will prevent or slow down the occurrence of diabetic nephropathy in a more efficient way and for treatments that are better tolerated and have fewer adverse effects.

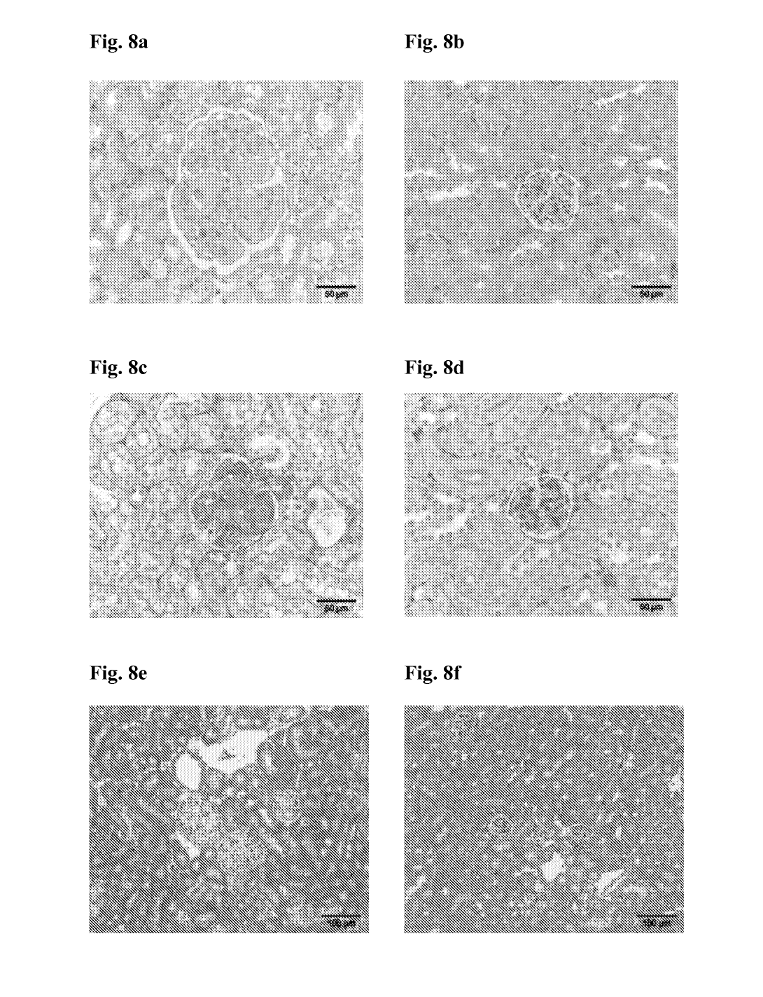

The following lesions were observed by histological evaluation in kidneys of 42 week old DIO mice vs. 25 week old DIO mice and 25 week old chow fed lean mice (see FIG. 8):
Minimal to slight glomerular hypertrophy (FIG. 8a vs. FIG. 8b)
Minimal to marked mesangial expansion (FIG. 8c vs. FIG. 8d)
Minimal to slight glomerulosclerosis (FIG. 8c vs. FIG. 8d)
Minimal to moderate infiltration of mononuclear inflammatory cells (FIG. 8e vs. 8f)

TABLE 2
Diabetic nephropathy in DIO mice
Incidence of relevant kidney changes

| | 25 wk old | 25 wk old | 42 wk old |
|---|---|---|---|
| | chow | DIO | DIO |
| Glomerular hypertrophy | 0/10 | 3/10 | 4/10 |
| Mesangial expansion | 3/10 | 8/10 | 10/10 |
| Glomerulosclerosis | 0/10 | 0/10 | 4/10 |
| Infiltration, mononuclear | 4/10 | 7/10 | 9/10 |

While there are shown and described presently preferred embodiments of the invention, it is to be distinctly understood that the invention is not limited thereto but may be otherwise variously embodied and practiced within the scope of the following claims.

1-12. (canceled)

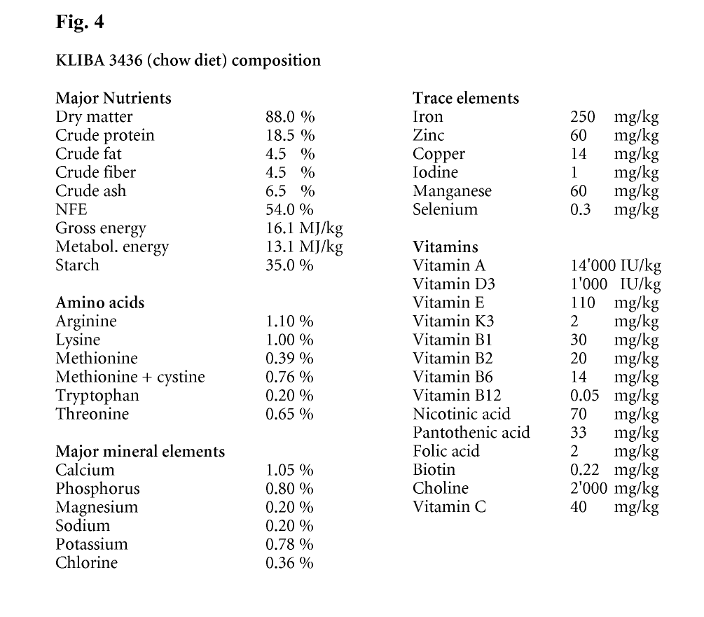

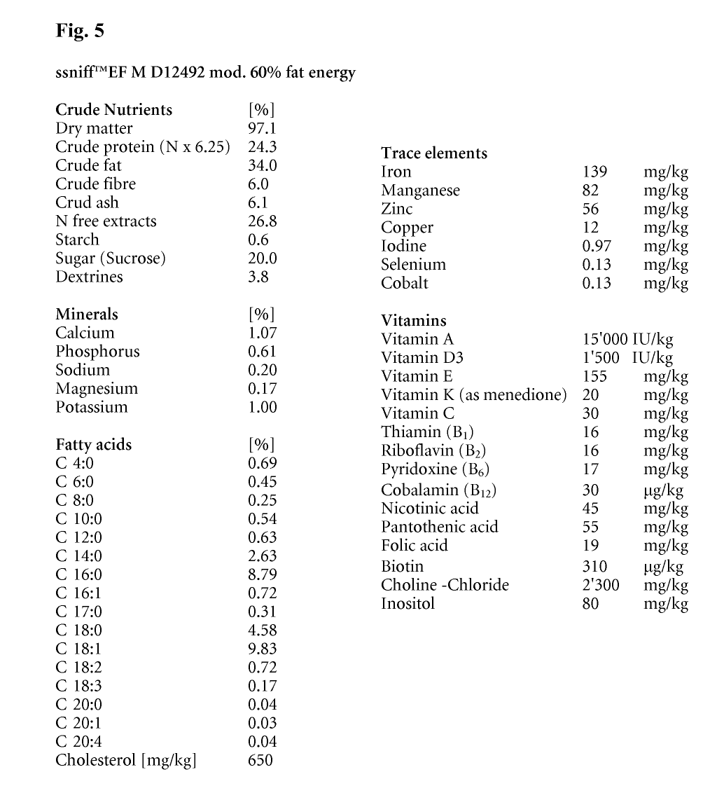

13. A method of producing a mouse model for insulin resistance, obesity and type 2 diabetes comprising
a) feeding a mouse a chow diet until the age of about 9 weeks, wherein the chow diet comprises KLIBA 3436 diet as described in FIG. 4, and
b) feeding the mouse of the age of about 9 weeks for about 20 to about 22 weeks with a high fat high sugar diet, wherein the high fat sugar diet comprises a SSNIFF D12492 modified 60% Kcal fat diet as described in FIG. 5.

14. The method of claim 13, wherein the mouse is a C57BL/6J mouse.

15. A method for producing a mouse model for complications of type 2 diabetes comprising
a) feeding a mouse a chow diet until the age of about 9 weeks, wherein the chow diet comprises KLIBA 3436 diet as described in FIG. 4, and wherein the mouse is a C57BL/6J mouse, and
b) feeding the mouse of the age of about 9 weeks for about 30 to about 32 weeks with a high fat high sugar diet, wherein the high fat sugar diet comprises a (custom made) SSNIFF D12492 modified 60% Kcal fat diet as described in FIG. 5.

16. A method for identifying and/or testing compounds for the treatment of type 2 diabetes and/or its complications, comprising the steps of administering a compound of interest to a mouse produced by the method of claim 13 or 15 and determining whether diabetes and/or at least one of its complications are reversed by said compound.
